(19) United States
(12) Patent Application Publication
(10) Pub. No.: US 2001/0044596 A1
(43) Pub. Date: Nov. 22, 2001

(54) Apparatus and method for treatment of vascular restenosis by electroporation

(76) Inventor: Ali Jaafar, Eden Prairie, MN (US)

Correspondence Address: Craig M. Gregersen, BRIGGS AND MORGAN, 2200 First National Bank Building, 332 Minnesota Street, Saint Paul, MN, 55101, US

(21) Appl. No.: 09/849,116

(22) Filed: May 4, 2001

Related U.S. Application Data
(60) Provisional application No. 60/202,536, filed on May 10, 2000.

(51) Int. Cl. A61M 31/00; A61N 1/30; A61B 5/04

(52) U.S. Cl. 604/103.01; 600/373; 604/509

(57) ABSTRACT

An apparatus and a method for reducing restenosis rate after balloon angioplasty is provided. The apparatus comprises a high voltage pulse generator and an intravascular catheter with electrodes adapted for delivery of electrical pulses to the media and adventitia of the treated segment of an artery. The amplitude, duration and number of the electrical pulses applied to the artery wall are sufficient to significantly deplete the population of smooth muscle cells, exuberant proliferation of which causes restenosis. To avoid possible fibrillation, the electric pulses are delivered during periods of depolarized state of myocardium.

### FIELD OF THE INVENTION

The present invention relates generally to electroporation of tissue and more particularly to apparatus and method for electroporation treatment of arterial tissue to prevent vascular restenosis after balloon angioplasty.

### BACKGROUND OF THE INVENTION

Atherosclerosis is a vascular disease that affects millions of people, often causing heart attacks and death. One aspect of this disease is stenosis, or the thickening of an artery wall, decreasing blood flow through the vessel. An angioplasty procedure has been developed to reopen stenotic arteries without resorting to a bypass surgery. In this procedure, a catheter carrying an expandable balloon is introduced into the diseased artery at the site of the blockage and then expanded to open the passage to increased blood flow. Frequently, an expandable stent is placed within the artery to support the arterial wall in the area where the blockage occurred. However, in a large number of cases, arteries become occluded again after an angioplasty procedure. This recurrent thickening of the vessel wall is known as restenosis. Restenosis frequently requires a second angioplasty and eventually bypass surgery. Bypass surgery is very stressful on the patient, requiring the chest to be opened, and presents risks from infection, anesthesia, and heart failure.

Restenosis occurs in nearly forty percent of all treated arteries. The cause of the restenosis is different from that of the original blockage. The original blockage, on one hand, is generally formed by plaque deposited over many years. The restenosis, on the other hand, is caused by the exuberant proliferation of smooth muscle cells (SMC) of the treated artery following the angioplasty procedure and can occur in as few as six months following the procedure.

FIGS. 10a, 10b, and 10c, respectively, generically illustrate in cross section: a typical artery following a routine balloon angioplasty; restenosis of an artery following a routine balloon angioplasty procedure; and in-stent restenosis of an artery following a routine balloon angioplasty procedure. Thus, in FIG. 10a, it will be observed that the artery A has been opened to allow substantially unimpeded blood flow therethrough. Arterial plaque that had previously obstructed the artery A is indicated at B. FIG. 10b, by comparison, illustrates how the rapid proliferation of smooth muscle cells C along the arterial wall has resulted in a substantial occlusion of the artery A. FIG. 10c depicts restenosis where the cell proliferation C has extended inwardly beyond the stent D implanted during the angioplasty procedure to support the arterial wall, once again leading to a substantial occlusion of the artery A.

There have been various methods and apparatus proposed for the treatment or prevention of restenosis, including further angioplasties and use of radiation or medication to kill the smooth muscle cells forming the arterial media. However, each of the proposed methods suffers from low efficacy, undesirable side effects and complications. Thus, new apparatus and methods for treating or preventing restenosis that do not suffer from these deficiencies would be desirable.

The biophysical phenomenon “electroporation” (EP) refers to the use of electric field pulses to induce microscopic pores—“electropores”—in cell walls or membranes. Depending on the parameters of the electric pulses, an electroporated cell can survive the pulse or die. The cause of death of an electroporated cell is believed to be a chemical imbalance in the cell, resulting from the fluid communication of the intracellular environment with the extra cellular environment through the pores.

The number and size of electropores produced by an electric field pulse depend on the product of the amplitude E and duration of the pulse t. Below a certain lower limit, no pores are induced at all. This lower limit is different for different cells, particularly, for cells of different sizes. The smaller the size, the higher product of the amplitude and duration must be to induce pore creation. As the product Et increases above the lower limit, so will the number of pores and their effective diameter. This number and size will continue to increase until an upper limit of the product Et is achieved.. Below the upper limit, cells survive pulsing and restore their viability thereafter. Above the upper limit of the product Et the pore diameters and numbers become too large for a cell to survive. The irreversibly chemically imbalanced cell cannot repair itself by any spontaneous or biological process and dies. Between the lower and the upper limits electroporation is known to be used for enhancement of drugs or genes delivery into cells.

In U.S. Pat. No. 5,273,525, issued to Gunter Hoffman, an apparatus for delivery macromolecules, such as genes, DNA or pharmaceuticals into cells of preselected tissue region or organ, is described. A modified syringe is provided for injecting a predetermined fluid medium, carrying the macromolecules. A signal generator is connected to the syringe for generating a predetermined electric signal. The syringe includes a pair of electrodes, which are connected to the signal generator for applying an electric field in the tissue. In order to make the cells membranes transiently permeable and viable, the field has a predetermined strength and duration between the lower and the upper limits. This method enhances the uptake of macromolecules and thus improves the therapeutic effect of the drug therapy.

In U.S. Pat. No. 5,944,710, issued to Sukhendu B. Dev et al, a method for local and intravascular drug delivery via electroporation is described. The method uses a catheter-based system for delivery of therapeutic agents, such as antiproliferative, anticoagulative and antiplatelet agents. The electroporation in this method is used for enhancement of drug delivery inside cells. The method is proposed for vascular applications such as deep vein thrombosis, peripheral arterial disease and cardiovascular restenosis. As a possible treatment for restenosis it suffers from several disadvantages. One of them is the absence of drugs available for efficient and safe killing of SMC inside an arterial wall. Another is the complexity of the method. It comprises two stages: the first is delivery of a toxic drug into the extra cellular space in the vessel wall and the second is electroporation. The cause of death of electroporated cells in this case is poisoning by the toxic drug. Combining in one intravascular apparatus an electrical and drug delivery devices is a serious engineering challenge, especially for coronaries, where a small size of the vessels combined with necessity to provide blood flow distally of the catheter makes it very difficult to accomplish, if possible at all.

It would be desirable to have an improved apparatus and method for reducing or preventing restenosis.

### SUMMARY OF THE INVENTION

It is an object of the present invention to provide a new and improved apparatus that is not subject to the foregoing disadvantages.

It is another object of the present invention to provide a method for the treatment of restenosis utilizing electroporation of smooth muscle cells.

It is another object of the present invention to eliminate or reduce the exuberant proliferation of smooth muscle cells following an angioplasty procedure.

The foregoing objects of the present invention are provided by a method and apparatus for reducing vascular restenosis after balloon angioplasty.

The present invention employs electroporation above the upper limit to inflict irreversible damage to smooth muscle cells at the angioplasty site and thus significantly deplete their population. Depletion of smooth muscle cell population slows down the process of their proliferation during healing and reduces restenosis. The method can be applied both in coronary and peripheral arteries. During electroporation treatment the product of the amplitude and duration of applied pulses are selected above the upper limit of electroporation of the cells. The cell killing by electroporation is a probabilistic process; that is, its result depends on the number of applied pulses. The electric field strength E, the duration of applied pulses t, and the number of pulses are selected to kill 99 to 99.9% of cells in the targeted volume.

An apparatus and method in accord with the present invention include a catheter with an expandable balloon for insertion into an artery. Once properly positioned at the desired location, the balloon is expanded. The balloon carries at least one electrode, with a second electrode being provided externally, or it may carry a pair of electrodes. During treatment electrical pulses of predetermined voltage and duration are applied between the electrodes. They cause corresponding pulses of electric current through the vascular tissue adjacent to the electrodes. The product of the electric field and the pulse duration at the inner surface of the vessel wall is selected to be above the upper electroporation limit for the smooth muscle cells. As the distance from the balloon surface into the vascular tissue increases, the electric field decreases, as well as the product of the electric field and pulse duration. The amplitude of the electric pulses is selected to provide the electric field and pulse duration product above the upper electroporation limit only at the depth about 1 mm or less into the vascular tissue. The objective of such electroporation treatment of the vascular wall is to kill smooth muscle cells in a cylindrical layer of the vascular tissue around the artery about 1 mm thick. Beyond this cylinder layer the vascular tissue is a subject for electroporation treatment with the electric field and pulse duration product under the upper electroporation limit, which means that the treatment causes only temporary reversible changes to that vascular tissue.

The foregoing objects of the invention will become apparent to those skilled in the art when the following detailed description of the invention is read in conjunction with the accompanying drawings and claims. Throughout the drawings, like numerals refer to similar or identical parts.

### BRIEF DESCRIPTION OF THE DRAWINGS

FIG. 1 is a schematic illustration of an electroporation system in accord with the present invention for treatment of restenosis.

FIG. 2a shows a time diagram for electroporation pulses applied in a regular periodic manner and FIG. 2b shows a time diagram of electroporation pulses synchronized with the T wave of a patient's ECG signal.

FIGS. 3a, 3b, and 3c are a schematic illustration of a bipolar electroporation balloon with ring electrodes in a longitudinal cross sectional view, a circumferential cross sectional view, and in a side elevation view, respectively.

FIG. 4a and 4b shows two bipolar electroporation catheters with multiple longitudinal and spiral electrodes, respectively, providing an electric field predominantly in the circumferential direction.

FIG. 5 shows a version of a bipolar electroporation catheter, in which two coaxial braids around the shaft of the catheter are used as conductors leading to multiple longitudinal electrodes on the balloon surface.

FIG. 6 is a schematic cross sectional, side elevation illustration of a unipolar electroporation catheter with the second electrode placed outside the patient's body.

FIGS. 7a, 7b, and 7c represent three stages of application of a unipolar electroporation balloon for simultaneous balloon angioplasty and electroporation treatment of an artery.

FIGS. 8a, 8b, and 8c illustrate schematically in cross sectional views three stages of the application of a unipolar electroporation catheter for treatment of the instent-restenosis.

FIG. 9 is a graph of a survival curve of electroporated cells as a function of amplitude of the electric pulses at a constant duration.

FIGS. 10a, 10b, and 10c respectively illustrate cross sectional views of: an artery after a balloon angioplasty procedure; restenosis of an artery following a balloon angioplasty procedure; and in-stent restenosis of an artery following a balloon angioplasty procedure

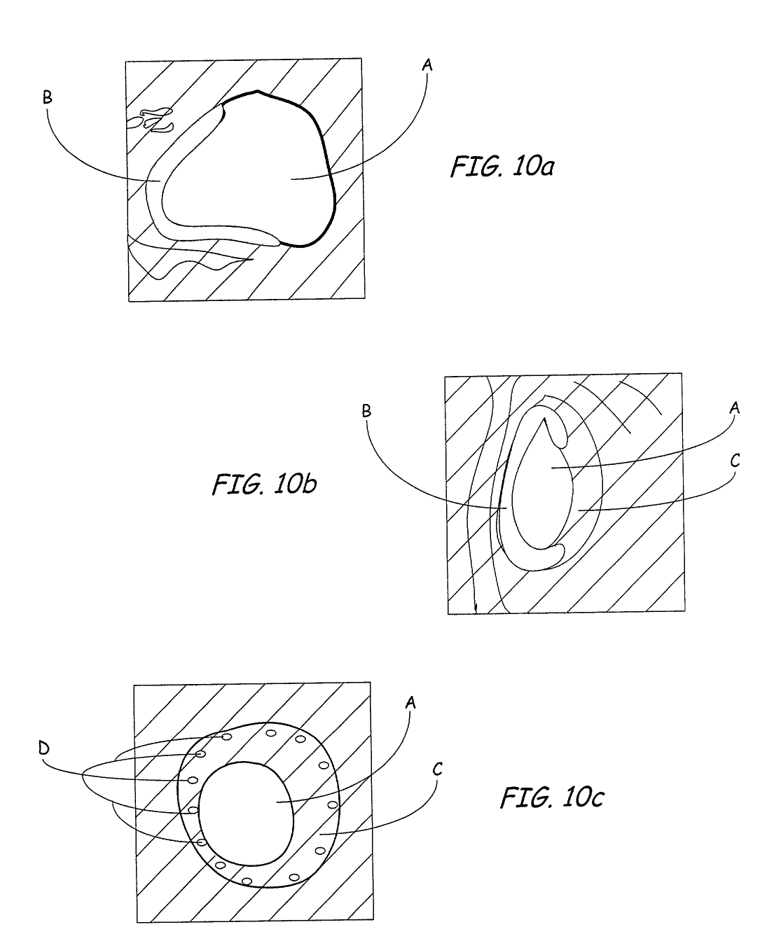

### DESCRIPTION OF THE PREFERRED EMBODIMENTS

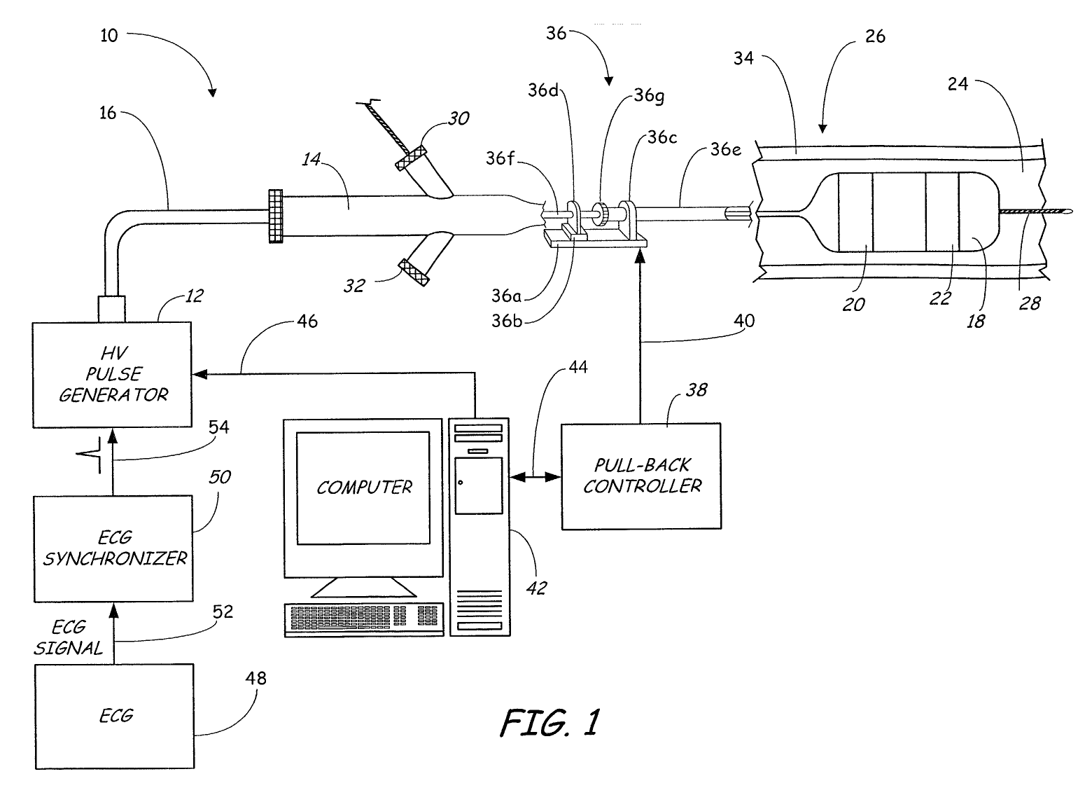

An electroporation system 10 in accord with the present invention and useful for the treatment of restenosis is shown in FIG. 1. The system 10 comprises a pulse generator 12 electrically connected to a catheter 14 by an appropriate electrical connector cable 16. An intravascular electroporation balloon 18 including electrodes 20 and 22 is delivered to the angioplasty site in the lumen 24 of an artery 26 over a guide wire 28. The catheter 14 includes at least a guide wire port 30 and may include a second or additional ports 32, which may be a port for saline or other fluid used for inflation of the balloon. Once inflated, the balloon 18 provides a close electric contact of its electrodes 20 and 22 with the vessel wall 34. The electroporation balloon 18 can be placed into the lumen 24 of an artery 26 after balloon angioplasty, or it can be functionally combined with an angioplasty balloon to provide electroporation treatment simultaneously with the dilatation of the artery 26 during the angioplasty procedure. In this case, the balloon 18 should possess the mechanical properties of a standard angioplasty balloon and, additionally, should have a set of electrodes for electroporation treatment.

In one embodiment of the present invention, shown in FIG. 1, a pullback or balloon retracting device 36 is provided, which is adapted to move the electroporation balloon 18 along the longitudinal extent of the arterial lesion. The pullback device 36 can be made in a number of different ways and each may be used in accord with the present invention provided that it gives the surgeon the requisite control over the movement of the balloon 18 along the longitudinal axis of the artery. Such a device may provide continuous or stepwise retraction. In the embodiment shown of a pullback device 36, such a device includes a body 36a and a carriage 36b slidable with respect to the body 36a. A distal adapter 36c in this case is mounted on the body 36a and a proximal adapter 36d is mounted on the carriage 36b. The distal adapter 36c is connected to the introducer sheath 36e, which does not move during treatment; the proximal adapter 36d is connected to the elongated shaft 36f of the electroporation catheter 14. A haemostatic valve 36g is provided to prevent leakage of blood from the introducer 36e, which is fluidly connected to the artery, outside the patient's body. The pullback device 36 transfers the motion of the slidable carriage 36b against the body 36a into the motion of the electroporation balloon 18 along the treated artery 26. It enables the use of a “one size fits all” catheter and balloon since it allows the treating lesions of various lengths. The device 36 is controlled by a pullback controller 38 over an appropriate signal line 40. The controller 38 is electrically connected to and is controlled by a computer over a line 44. This, or another computer can be provided to control the number, amplitude, polarity and duration of electrical pulses applied to the artery through the electrodes 20 and 22. In the embodiment shown in FIG. 1, such control is provided by computer 42 to generator 12 over a line 46.

In one embodiment of the present invention, the pulse generator 12 is synchronized with the heartbeat of a patient. An electrocardiograph 48 is provided to provide a signal indicative of the electrical status of a patient's heart to a synchronizer 50 over a signal line 52. The synchronizer 50 is provided to synchronize the pulsing of the vessel with the electrodes 20 and 22 with the beating of the patient's heart. The synchronizer forms a triggering pulse, coinciding with the T wave of the electrocardiogram of a patient's heart produced by the electrocardiograph 48, which it provides to the generator 12 over the appropriate line 54. Those familiar with electrocardiography will recognize that the electrical output of the heart during a beat is characterized by a wave form designated a PQRST wave. During the T wave portion, the myocardium of the heart is depolarized and insensitive to electrical pulses. Providing the electroporation treatment during this portion of the heartbeat cycle prevents the electroporation pulses from creating a fibrillation—or rapid and irregular beating—of the heart.

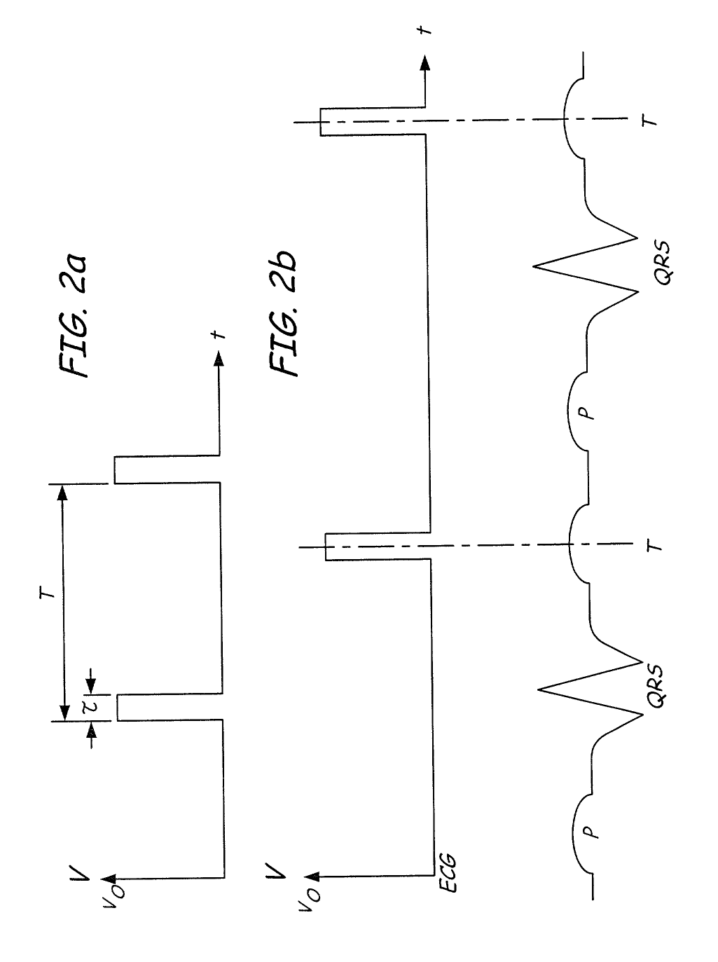

FIG. 2 depicts time diagrams of electroporation pulses. FIG. 2a shows a simple operation that is not synchronized with the heart beat. FIG. 2a represents the application of an electrical pulse to a tissue at a regular interval. The use of such a regular periodic pulse application is most appropriate for treatment of peripheral blood vessels where there is no concern for interrupting the patient's normal heartbeat. FIG. 2b shows electroporation pulses that are synchronized with the T wave portion of the cardiogram, which as noted is more appropriate for coronary arteries.

There are two different designs of the electroporation balloons. These two designs use bipolar and unipolar electrode systems. In the first case, both electrodes are mounted on a balloon and placed in the same artery. In the second case, one electrode is mounted on a balloon in the artery, and the other is placed outside the patient's body on the skin close to the first electrode.

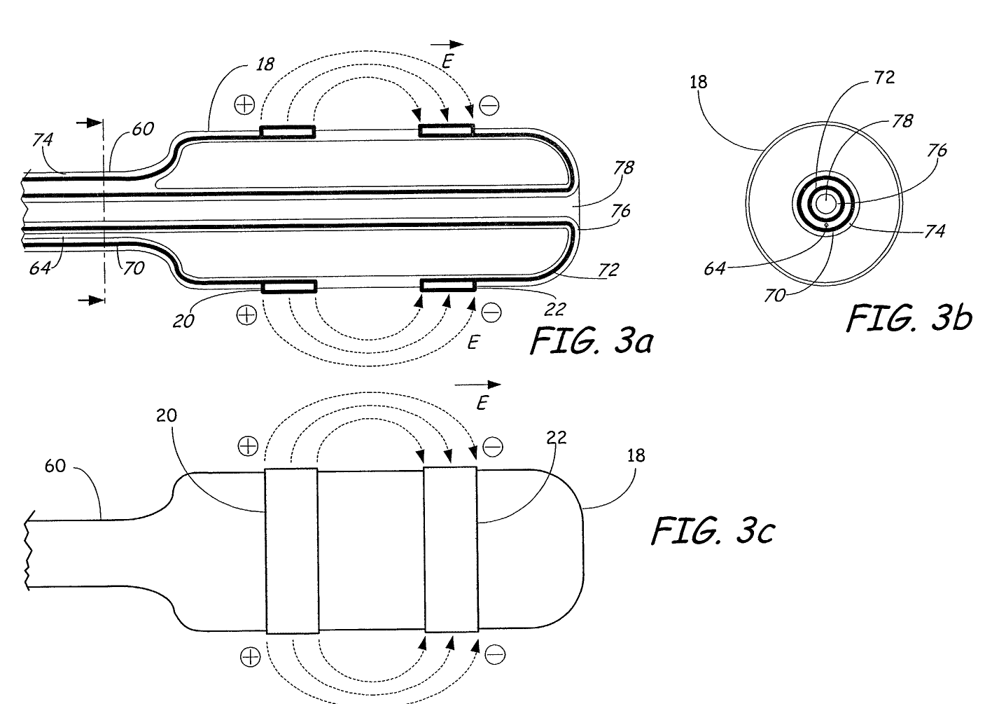

A bipolar electroporation balloon is shown in FIGS. 3a, 3b, and 3c. An elongated shaft 60 of an intravascular catheter, such as catheter 14 is illustrated, at the distal end of which a balloon 18 is secured. A channel 64 in the shaft 60 leads to the balloon 18 and serves to provide a fluid passage from an external fluid source, typically a source of saline, for the balloon's inflation. The balloon 18 supports a pair of electrodes 20 and 22, which are electrodes, electrically connected to the pulse generator by conductors 70 and 72, respectively. Insulating coatings 74 and 76 over conductors 70 and 72 prevent electric current between conductors anywhere but between electrodes 20 and 22 in the treatment area. A channel 78 inside the shaft serves for delivering the catheter over a guide wire to the angioplasty site.

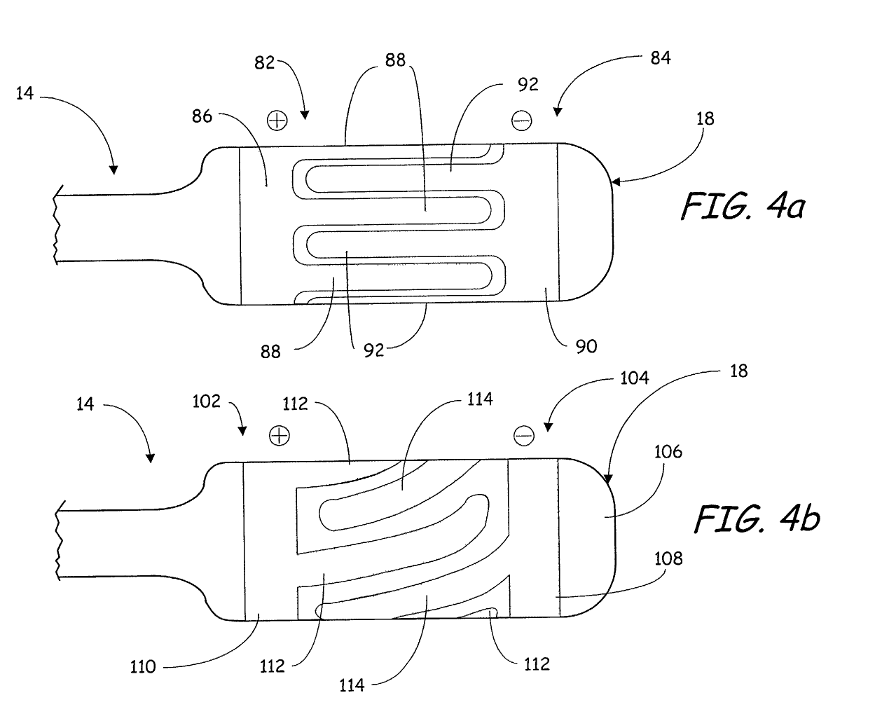

In FIG. 4 two bipolar electroporation catheters with longitudinal electrodes are shown. In the catheter 14 shown in FIG. 4a the electrodes 82 and 84 are positioned along the axis of the balloon 18. It will be observed that electrode 82 comprises a circular band 86 with a plurality of distally extending, spaced apart members 88. Electrode 84 similarly comprises a circular band 90 with a plurality of proximally extending, spaced apart members 92. The members 88 and 92 are interdigitated such that an member of one polarity is sandwiched between two members of the opposite polarity. The electrodes 82 and 84 provide an electric field predominantly in the circumferential direction.

FIG. 4b illustrates a catheter with electrodes 102 and 104 positioned in a spiral manner about the outer surface 106 of the balloon 18. Each electrode 102 and 104 includes a circular band 108, 110 respectively. From electrode band 108 a plurality of electrode members 114 extend distally and spirally about surface 106. From electrode band 110 a plurality of electrode members 112 extend proximally and spirally about surface 106. The electrode members 112 and 114 are interdigitated such that a member of one polarity is sandwiched between two members of the opposite polarity. Near the electrodes the electric field is not circumferential. The spiral version of the electrode configuration allows treatment of the whole vessel wall with a circumferential field by pulling the catheter back by a step, equal to the pitch of the spiral.

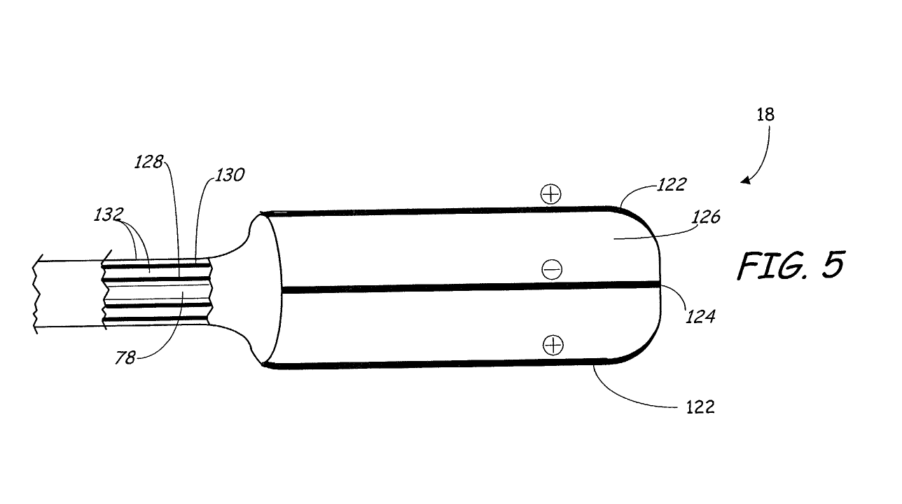

An electroporation bipolar catheter with coaxial conductors placed over the catheter shaft is shown in the FIG. 5. Multiple longitudinal electrodes 122 and 124, positive and negative, secured on the balloon 18, provide a circumferential electric field. Two coaxial conductors 128 and 130 provide electrical connection of the electrodes of both polarities to the pulse generator. An insulator 132 is provided for the conductors.

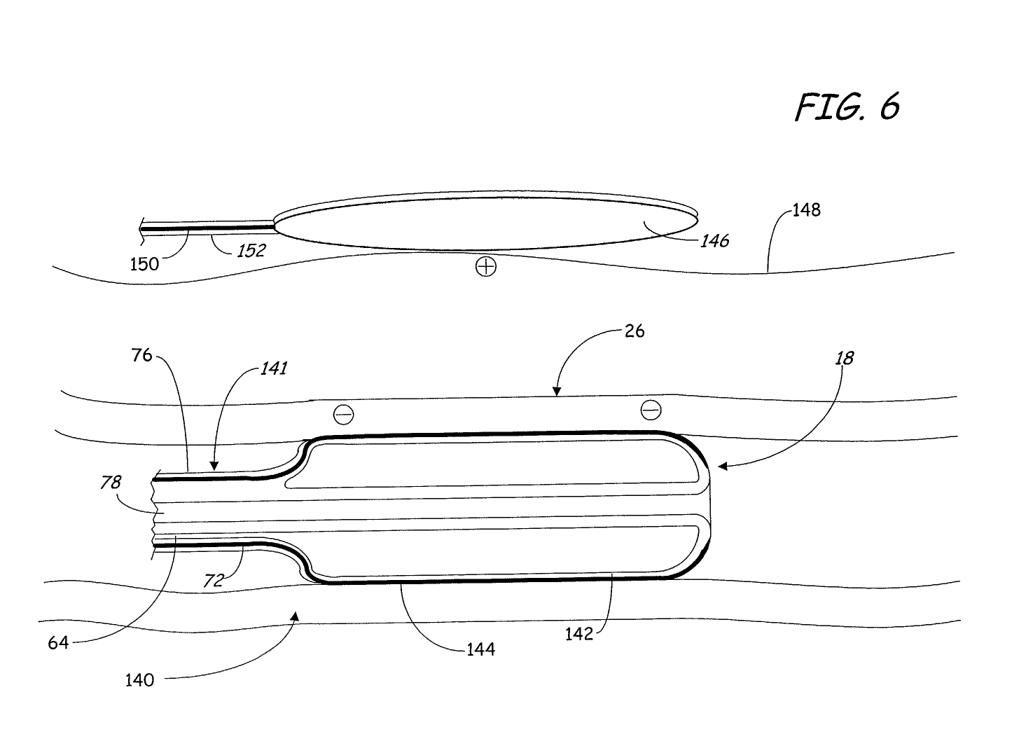

In FIG. 6 through 8 different versions of the electroporation unipolar catheters in accord with the present invention and their applications are shown.

FIG. 6 illustrates in a partial cross sectional view a unipolar intravascular catheter 140. An inflatable balloon 18 is shown at the end of the elongated shaft 141 of the catheter. A low profile channel 64, fluidly connected to the balloon 18, serves for its inflation. All or substantially all of the outside surface 142 of the balloon 18 is metallized to form and perform the function of the intravascular electrode 144. The electroporation balloon is delivered to an angioplasty site over the guide wire lumen 78. The second electrode 146 is positioned outside the patient's body 148, and connected to the pulse generator by a conductor 150, insulated with an insulator 152.

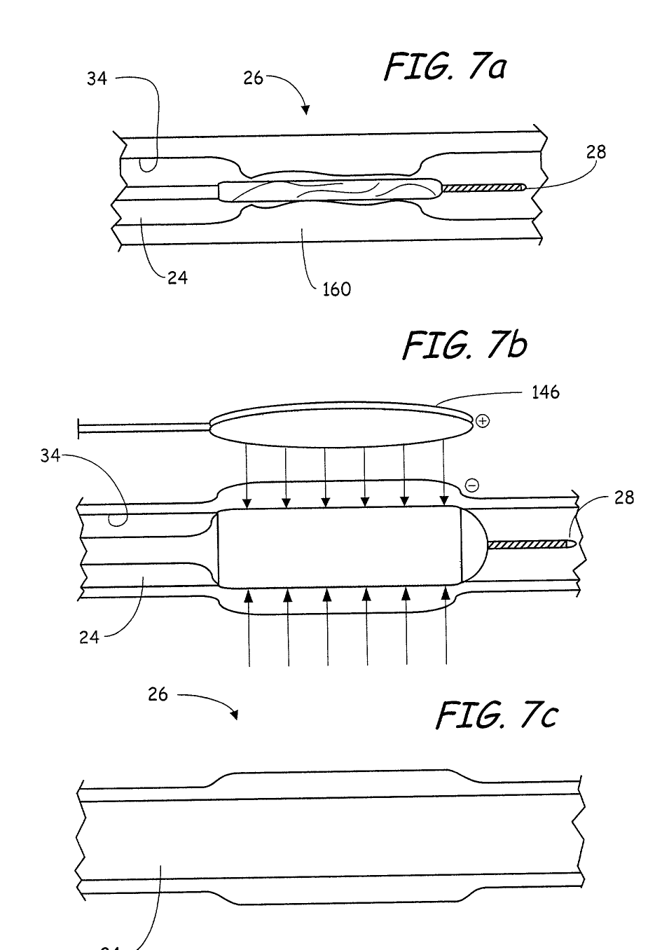

FIGS. 7a to 7c schematically present three consecutive stages of a percutaneous translumenal coronary angioplasty (PTCA) intervention, combined with an electroporation anti-restenosis treatment for a new lesion using a unipolar electrode system. In FIG. 7a, a double function (dilatation plus electroporation) balloon 18 is delivered over the guide wire 28 to a stenotic site 160 in an artery 26. It will be observed that the artery is shown as being partially occluded by the build up of arterial plaque over the years. In FIG. 7b, the balloon 18 has been expanded to dilate the artery 26 and electroporation treatment to the stenotic site 160 has begun with the use of the external electrode 146. The expansion of the balloon 18 has enlarged the lumen 24 of the artery 26 in the area of the stenosis 160 such that it has substantially the same cross sectional area as the artery does both upstream and downstream of the blockage 160. It will be understood that this electrode 146 is disposed externally of the patient's body and that the patient's body has been omitted from the Figure for clarity of illustration. FIG. 7c illustrates schematically the artery after the dilatation and electroporation treatment.

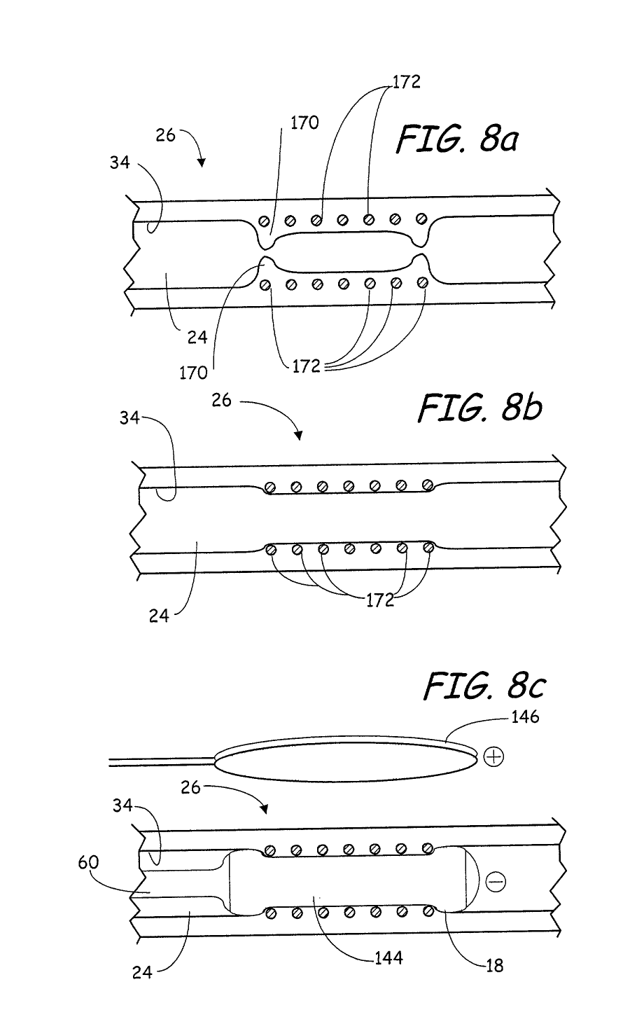

FIGS. 8a to 8c present an “in-stent-restenosis” treatment. In FIG. 8a a lesion 170 before intervention is shown. The stent 172 has been overgrown by the exuberant proliferation of smooth muscle cells—the lesion 170 targeted for treatment. The stent 172 has been illustrated schematically to be a scaffold structure. It will be appreciated that the present invention is not limited to the particular structure of any particular stent and that the present invention finds use with any stent of any construction. Referring to FIG. 8b, the artery 26 is illustrated after removal of the arterial obstruction or hyperpalasia 170 in the stent by any tool capable of such removal action (for example, by a rotablator). That is, the surgeon has first taken steps to remove the obstruction and to once again open the artery 26 to full or nearly full blood flow. The lumen 24 has been substantially opened to blood flow. In FIG. 8c an electroporation treatment with a unipolar electroporation balloon is shown. Similar to the previously described Figures, a catheter having a balloon 18 is inserted into the lumen 24 of the artery 26 and disposed within or substantially within the lumen 24 at the site of the lesion 170. As illustrated in FIG. 8c, the balloon 18 includes a unipolar electrode. Once properly positioned, the balloon will be expanded so as to substantially obstruct the lumen passage and to place the electrode 144 substantially adjacent the arterial wall 34. Subsequently to the expansion of the balloon 18, the second electrode 146 will be properly positioned relative thereto and the proper electrical pulses will be applied through the balloon 18 to the arterial wall 34.

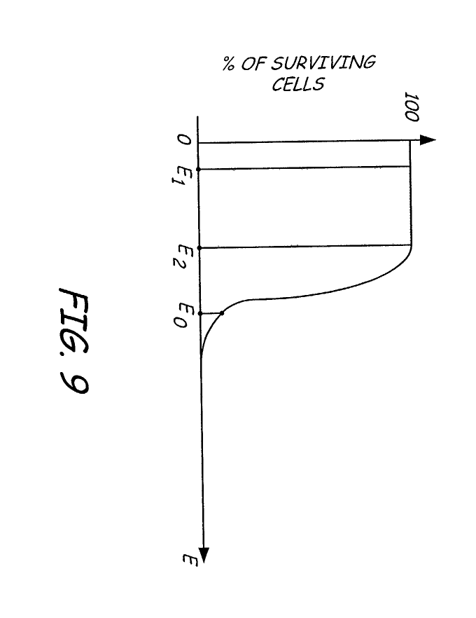

In FIG. 9a survival curve for electroporated cells as a function of an applied electric field is plotted. The duration of pulses is assumed to be constant. While the amplitude of an electric pulse remains below the electroporation induction or lower limit E1, nothing happens to the cells. Above this limit, but below the upper electroporation limit E2, reversible pores are induced in the cell membranes and the cells survive the electroporation pulses. Above the upper electroporation limit E2, the survival curve starts going down and approaches zero at some high value of electric field E. The nature of the curve is probabilistic, its spread depends on the nature of the cells, their differences in sizes and angular positions relatively to the direction of the electric field. The same survival results can be achieved by applying not only one high amplitude pulse, but also a number of pulses of lower amplitudes or duration. The only condition is that the operating amplitude E0 should be above the upper electroporation limit of electroporation E2.

In muscular arteries smooth muscle cells are arranged in spiral bundles around the artery. The pitch of this spiral is small, so the cells are positioned with their length almost in a tangent direction. The length of smooth muscle cells is around 60 microns while the width about 3 microns. To reach the upper limit of electroporation for a given cell, a voltage around 1-1.5 volts should be applied across the cell. Where cells vary in size in different directions, then different voltages will need to be applied dependent upon the desired direction of the applied field. If a set of electrodes on an electroporation balloon is adapted for application of electric field in a circumferential direction, then the electric field, that has to be applied to kill SMC, should be in the range of 175 to 300 V/cm. For killing cells by electric field, applied along their width, approximately 20 times as high field is required, 3500 to 6000 V/cm. The duration of the pulses can be chosen between a fraction of a microsecond to tens or hundreds milliseconds. To accumulate a high Et product, defining low survival rate, hundreds of pulses can be applied to the same site.

The method of treatment of an angioplasty site to prevent restenosis consists of providing a high voltage pulse generator with pulses of predetermined amplitude, duration and polarity, and an intravascular balloon catheter having at least one pair of electrodes on its balloon, with the electrodes being electrically connected to the aforesaid generator. The subsequent steps include the introduction of the catheter into a treated artery to a predetermined angioplasty site, inflation of the balloon and the application of a predetermined number of pulses, synchronized or not synchronized with the depolarized state of the heart's myocardium. In different implementations of the method, the catheter can be pulled back to provide better uniformity of electroporation treatment of the vessel wall. The polarity of its electrodes and amplitudes of the pulses can vary during the electroporation treatment of the vessel. The present invention further contemplates the use of an inflatable electroporation balloon having the appropriate mechanical properties for performing a balloon angioplasty procedure to be used with an angioplasty being performed prior to the electroporation treatment to prevent the onset of restenosis.

During a treatment, as noted previously, the voltage, current, and duration of the applied pulses will be strictly controlled to reduce the likelihood of injury to a patient. Typically, an electrical pulse generator in accord with the present invention will generate pulses in the range of about 100 volts to about 10000 volts to provide an electric field on the smooth muscle cells above the upper electroporation limit. Within an artery, such pulses will desirably have a voltage in the range of about 1 to about 5 volts across the smooth muscle cells of an artery so as to be able to irreversibly damage them. The preferred duration of the electrical pulses will fall within the range of about 0.1 microsecond to about 10 milliseconds.

In another implementation of the electroporation treatment with a unipolar intravascular electrode, instead of a voltage control of the electroporation pulses, a current control of the pulsed electric current through the surrounding tissue can be used. Information about the electrode geometry (radius and length), the electric current density on the electrode surface and the tissue resistivity allows an operator to calculate the electrical field in the tissue near the electrode, and control its value exactly at the necessary level. The necessary level means that in the target area (media and adventitia) the operating field E0 should be above the upper A electroporation limit to be able to kill smooth muscle cells. At the same time, beyond the target area, in the myocardium, the field and the corresponding current density should be bellow the upper electroporation limit to cause only reversible change to the cells. Electrical high voltage pulsers with current limiting circuits can be used in this version of the electroporation systems for restenosis to prevent accidental application of high current to a patient.

Contrary to ionizing radiation, electroporation does not inflict any damage to DNA of the treated cells. The cells are killed by rupture of their membranes, separating the inner cellular space from the extra cellular environment. The cause of death is an irreversible biochemical imbalance in the cells. Survived cells are normal, without any hidden damage to their DNA or unknown long term consequences. Electroporation treatment, contrary to ionizing radiation, can be used multiple times for the same angioplasty site.

The present invention having thus been described, other modifications, alterations, or substitutions may now suggest themselves to those skilled in the art, all of which are within the spirit and scope of the present invention. It is therefore intended that the present invention be limited only by the scope of the attached claims below.

What is claimed is:

1. An apparatus for reducing restenosis after a balloon angioplasty procedure, comprising:
an electrical pulse generator, said generator being provided for producing a predetermined number of pulses having a predetermined voltage, current, and duration;
an intravascular catheter having proximal and distal ends and including an inflatable balloon at its distal end, said balloon having on its surface at least one electrode electrically connected to said pulse generator.

2. The apparatus according to claim 1 and further comprising a pull back device for providing predetermined retraction of said inflatable balloon along the vessel.

3. The apparatus of claim 2 wherein said pull back device provides stepwise retraction of said inflatable balloon.

4. The apparatus of claim 1 and further comprising an ECG apparatus and a synchronizing device, said ECG apparatus and synchronizing device being operatively connected to said generator and providing synchronization of the electrical pulses generated by said pulse generator with at least one depolarized phase of a patient's myocardium.

5. The apparatus of claim 1 wherein said balloon has at least a set of two electrodes of different polarity disposed on the outer surface of said balloon in such a manner that the electric current passes mainly through the vessel wall.

6. The apparatus of claim 1 wherein said balloon has a first electrode on the outer surface of said balloon and further including a second electrode of the opposite polarity to said first electrode for placement on the outside surface of a patient's body during an operative procedure.

7. The apparatus of claim 6 wherein said first electrode covers substantially the entire outer surface of said balloon.

8. The apparatus of claim 1 wherein said electrical pulse generator generates pulses in the range of about 100 volts to about 10000 volts to provide an electric field on the smooth muscle cells above the upper electroporation limit.

9. The apparatus of claim 8 wherein said pulses have a voltage in the range of about 1 to about 5 volts across the smooth muscle cells of an artery so as to be able to irreversibly damage them.

10. The apparatus of claim 1, in which said inflatable electroporation balloon is used simultaneously as a dilatation balloon.

11. The apparatus of claim 1 wherein the duration of the electrical pulses falls in the range of about 0.1 microsecond to about 10 milliseconds.

12. The apparatus of claim 1 wherein the amplitude of the pulsed is controlled by a current limiting circuit.

13. The apparatus of claim 1 and further including an electrical connector disposed proximally on said catheter and an elongated conductor extending between said proximal connector of the catheter and said electrode on the distal balloon, wherein said electrical connectors are coaxial with the catheter shaft.

14. The apparatus of claim 1 wherein said balloon has at least a set of two electrodes of different polarity disposed on the outer surface of said balloon, each of said electrodes comprising conductive ribbons extending along the longitudinal axis of said balloon.

15. The apparatus of claim 1 wherein said balloon has at least a set of two electrodes of different polarity disposed on the outer surface of said balloon, wherein each of said electrodes includes a conductive band substantially encircling, wherein a first of said electrodes includes at least one conductive member extending distally from said first electrode band and a second of said electrodes includes at least one conductive member extending proximally from said second electrode band.

16. The apparatus of claim 15 wherein each said electrode includes a plurality of conductive members extending from its conductive band and wherein said members are interdigitated.

17. The apparatus of claim 16 wherein said conductive members extend longitudinally along the axis of said balloon.

18. The apparatus of claim 16 wherein said conductive members extend spirally about the outer surface of said balloon.

19. A method of reducing restenosis after angioplasty, comprising:
providing an electrical pulse generator;
providing an inflatable intravascular balloon having at least one electrode on its surface, the second electrode is provided on the same balloon or on the patient's skin outside the body;
delivering a predetermined number of electrical pulses generated by the pulse generator to the electrodes, said pulses providing the electric field to the smooth muscle cells above the upper electroporation limit.

20. A method according to claim 19, in which the amplitude, duration and number of the applied pulses kill 99.0% to 99.9% of smooth muscle cells in the media and adventitia of the blood vessel at the angioplasty site.

21. A method according to claim 19 wherein the amplitude of the pulses is controlled by a current limiting circuit.

22. A method according to claim 19 wherein the electrical pulses applied to smooth muscle cells are synchronized with a depolarized phase of a patient's myocardium.

23. A method according to claim 19 wherein the dilatation and electroporation treatment of the angioplasty site are performed simultaneously using the same balloon.
